(19) United States
(12) Patent Application Publication
(10) Pub. No.: US 2025/0270491 A1
(43) Pub. Date: Aug. 28, 2025

(54) SMART ALGAE SHADES FOR WATER EVAPORATION REDUCTION FROM ALGAL OPEN PONDS

(71) Applicant: SAUDI ARABIAN OIL COMPANY, Dhahran (SA)

(72) Inventors: Ameerah Mohammed Madani Bokhari, Thuwal (SA); Amr I. Abdel-Fattah, Dhahran (SA); Khalid Hazazi, Dhahran (SA)

(73) Assignee: SAUDI ARABIAN OIL COMPANY, Dhahran (SA)

(21) Appl. No.: 18/589,153

(22) Filed: Feb. 27, 2024

(51) Int. Cl. C12M 1/09; C12M 1/00; C12M 1/04; C12M 1/34

(52) U.S. Cl. CPC C12M 23/56; C12M 23/18; C12M 23/22; C12M 23/24; C12M 41/26; C12M 41/34; C12M 41/44

(57) ABSTRACT

A system for algae cultivation includes an algae shade having a gas permeable transparent sheet coated with a gas permeable membrane layer. The gas permeable membrane layer is coated on a top side of the gas permeable transparent sheet and a bottom side of the gas permeable transparent sheet respectively, and the gas permeable membrane layer has a thickness of from 1.0 nm to 10,000 nm. A method of cultivating algae using the algae shade is also provided.

### BACKGROUND

Algal open ponds are among the most common efficient systems to cultivate algae. Algae in open ponds conduct photosynthesis for algae growth. Light, water, and carbon dioxide are used for photosynthesis and oxygen may be produced in the reaction process of the photosynthesis. However, algal open ponds suffer high water evaporation that increases salinity of the pond water which affects algal growth and productivity.

### SUMMARY

Embodiments herein are directed toward algae shades that may transfer light and may allow uptake of carbon dioxide and expulsion of oxygen. Furthermore, such algal shades may reduce water evaporation of the algal open pond and may be advantageous in increasing photosynthesis and growth of algae.

In one aspect, embodiments disclosed herein relate to a system for algae cultivation. The system comprises an algae shade comprising a gas permeable transparent sheet coated with a gas permeable membrane layer. The gas permeable membrane layer is coated on a top side of the gas permeable transparent sheet and a bottom side of the gas permeable transparent sheet respectively, and the gas permeable membrane layer has a thickness of from 1.0 nm to 10,000 nm.

In another aspect, embodiments disclosed herein relate to a method for algae cultivation, comprising introducing a system for algae cultivation into an algal pond, and cultivating algae in the algal pond. The system comprises an algae shade comprising a gas permeable transparent sheet coated with a gas permeable membrane layer. The gas permeable membrane layer is coated on a top side of the gas permeable transparent sheet and a bottom side of the gas permeable transparent sheet respectively, and the gas permeable membrane layer has a thickness of from 1.0 nm to 10,000 nm.

In yet another aspect, embodiments disclosed herein relate to a method for algae cultivation comprising introducing a system of algae cultivation into an algal pond, monitoring the at least one parameter selected from salinity, pH, water level, and dissolved gas concentration of water in the algal pond, adjusting the at least one parameter selected from salinity, pH, water level, and dissolved gas concentration of water in the algal pond based on results of the monitoring, and cultivating algae in the algal pond. The system comprises an algae shade comprising a gas permeable transparent sheet coated with a gas permeable membrane layer and one or more sensors to monitor at least one parameter selected from salinity, pH, water level, and dissolved gas concentration of water. The gas permeable membrane layer is coated on a top side of the gas permeable transparent sheet and a bottom side of the gas permeable transparent sheet respectively, and the gas permeable membrane layer has a thickness of from 1.0 nm to 10,000 nm. This summary is provided to introduce a selection of concepts that are further described below in the detailed description. This summary is not intended to identify key or essential features of the claimed subject matter, nor is it intended to be used as an aid in limiting the scope of the claimed subject matter. Other aspects and advantages of the claimed subject matter will be apparent from the following description and the appended claims.

### BRIEF DESCRIPTION OF THE DRAWINGS

FIG. 1 is a cross sectional view of the smart algae shade containing a gas permeable transparent sheet coated with a gas permeable membrane layer according to embodiments herein.

FIGS. 2A-2B are schematic drawings of a shade system according to embodiments herein.

FIG. 3 is a schematic drawing of the shade system of FIG. 2B disposed in an algal pond to reduce water evaporation by covering at least a portion of the surface of the algal pond water according to embodiments herein.

### DETAILED DESCRIPTION

In the following Detailed Description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Algae in open ponds conduct photosynthesis for algal growth, which is a process that converts light energy to chemical energy. Light, water, and carbon dioxide are used for photosynthesis and oxygen is produced in the reaction process of the photosynthesis. However, algal open ponds suffer high water evaporation that increases salinity of the pond water and affects algal growth. In one or more embodiments, a smart algae shades (SAS) system may transfer light, may allow uptake of carbon dioxide and expulsion of oxygen, and may reduce water evaporation of the algal pond which increases photosynthesis and leads to the increase of algae growth and productivity.

Embodiments disclosed herein generally relate to a system, a composition, and a method for algae cultivation that reduces water evaporation from algae open ponds without negatively impacting the photosynthesis within the pond. Embodiments herein provide a transparent system for “shading” a pond. The system occupies surface area of the pond to limit evaporation while permitting light to pass through the system so as to not interfere with the photosynthesis process. Embodiments herein may also provide for monitoring of conditions within the pond.

### Gas Permeable Transparent Sheet

In one or more embodiments, the system includes a smart algae shade containing a gas permeable transparent sheet coated with a gas permeable membrane layer. The membrane layers are also transparent. The system, including the smart algae shade containing the gas permeable transparent sheet and membrane layers, allows visible light to pass through the sheet/membrane layers into the pond water such that light used for algal photosynthesis reaches the algae in the pond. Furthermore, in one or more embodiments, the transparent sheet may be permeable to gas which allows uptake of carbon dioxide and expulsion of oxygen to increase photosynthesis. Thus, the composition of the transparent sheet is tuned such that it is transparent to light and selectively permeable to gases. The term “transparent” used herein refers to a sheet and membrane layer that may have a total light transmittance of 26 μmol photons·m⁻²·s⁻¹ (micromoles of photons per meter squared per second) to 400 μmol photons·m⁻²·s⁻¹. For example, the gas permeable transparent sheet of one or more embodiments may have a total light transmittance in a range having a lower limit of any one of 26, 28, 30, 32, 34, 36, 38, 40, 42, 44, 46, 48, 50, 100, 110, 120, 130, 140, 150, 200, 250, and 300 μmol photons·m⁻²·s⁻¹, and having an upper limit of any one of 100, 150, 200, 250, 300, 310, 320, 340, 350, 360, 370, 380, 382, 384, 386, 388, 390, 392, 394, 396, 398, and 400 μmol photons·m⁻²·s⁻¹, where any lower limit may be paired with any mathematically compatible upper limit.

In one or more embodiments, the composition of the gas permeable transparent sheet includes a transparent biocompatible polymer. As used herein, the term “biocompatible polymer” means a polymer that does not negatively impact the microalgae/biomass growth in the ponds. Examples of the transparent biocompatible polymers useful in embodiments herein include, but are not limited to, an acrylamide, a polyurethane, a gelatin, an agar, a collagen, an alginate, a carrageenan, a polysiloxane, or a combination thereof. More specifically, the examples of biocompatible polymers useful in embodiments herein include, but are not limited to, polyhydroxyalkanoates (PHA), polylactic acid (PLA), and chitosan. In one or more particular embodiments, the biocompatible polymer may be a photo-crosslinkable resin. Examples of the photo-crosslinkable resin useful in embodiments herein include, but are not limited to, polyvinyl alcohol (PVA), coumarin and its derivatives such as 7-hydroxy-4-methylcoumarin (HMC), biobased acrylate photopolymer resins, methacrylates and epoxides.

It is generally known that plants and algae include chlorophyll which performs photosynthesis in the presence of light. Chlorophyll strongly absorbs red light which enhances photosynthesis. Therefore, in addition to being transparent, permitting light to pass through the shade into the pond may also enhance photosynthesis by converting light being transmitted through the shade to more favorable frequencies for photosynthesis. In one or more embodiments, the composition of the gas permeable transparent sheet may further include a light frequency-shifting material. The light frequency-shifting materials useful in embodiments herein may include, but are not limited to, a quantum dot, a fluorescent protein, and combinations thereof. In particular embodiments, the quantum dots may be incorporated into a gas permeable membrane layer of the shade system.

The light frequency-shifting materials disclosed herein may act as light frequency-shifting agents that absorb light proportional to frequencies between ultraviolet ray and green light, and emit red light such that it may increase photosynthesis of algae in an algal pond. Specifically, in one or more embodiments, the light frequency-shifting material may exhibit an effective stokes shift of about 75 nm that is effective for photosynthesis.

The gas permeable transparent sheet has suitable total light transmittance such that the light use for photosynthesis of algae may pass through the gas permeable transparent sheet. In one or more embodiments, the gas permeable transparent sheet including a transparent biocompatible polymer may have a total light transmittance of 26 μmol photons·m⁻²·s⁻¹ to 400 μmol photons·m⁻²·s⁻¹.

As noted above, the gas permeable transparent sheet has a gas permeability suitable for carbon dioxide uptake and oxygen expulsion such that it may be advantageous to increase photosynthesis and growth of algae in algal ponds.

In one or more embodiments, the smart algae shades (including the gas permeable transparent sheet and gas permeable membrane layer) may have a thickness of from 1 μm to 10 μm. For example, the smart algae shades of one or more embodiments may have a thickness in a range having a lower limit of any one of 1, 1.5, 2, 2.5, 3, 3.5, 4, 4.5, 5, 5.5, 6, 6.5, and 7 μm, and having an upper limit of any one of 4, 4.5, 5, 5.5, 6, 6.5, 7, 7.5, 8, 8.5, 9, 9.5, and 10 μm, where any lower limit may be paired with any mathematically compatible upper limit.

### Gas Permeable Membrane Layers

As discussed above, algae use photosynthesis to convert light energy to chemical energy for algal growth. Carbon dioxide is one of the reactants for photosynthesis and oxygen is a byproduct produced in the process of photosynthesis. Nitrogen also plays a vital role in algal growth. In order to perform efficient photosynthesis, a system that is highly selective to carbon dioxide and oxygen may be advantageous. Therefore, in one or more embodiments, the gas permeable transparent sheet may be coated with a gas permeable membrane layer.

Specifically, in one or more embodiments, a gas permeable membrane layer is coated on a top side of the gas permeable transparent sheet and a bottom side of the gas permeable transparent sheet respectively. The “top side” of the gas permeable transparent sheet of the present disclosure refers to a surface of the gas permeable transparent sheet facing towards the atmosphere and away from the pond water. The “bottom side” of the gas permeable transparent sheet of the present disclosure refers to a surface of the gas permeable transparent sheet facing or in contact with the surface of algal pond water.

As noted above, the membrane layer has a gas permeability suitable for carbon dioxide uptake and oxygen expulsion such that it may be advantageous to increase photosynthesis and growth of algae in algal ponds. In one or more embodiments, the gas permeable membrane layer is highly selective to carbon dioxide and oxygen such that the gas membrane layer allows 1-way diffusion of carbon dioxide (uptake) and 1-way diffusion of oxygen (expulsion). For example, the gas permeable transparent sheet on the top side of the gas permeable transparent sheet allows uptake of carbon dioxide and the gas permeable transparent sheet on the bottom side of the gas permeable transparent sheet allows expulsion of oxygen.

The uptake of carbon dioxide and expulsion of oxygen depend on the molecular size of carbon dioxide and oxygen and solubility of carbon dioxide and oxygen to water, in addition to natural selection of algae biomass for carbon dioxide over oxygen in pond water.

The membrane layer may have a plurality of pores with a pore size that can transfer carbon dioxide and oxygen. It is generally known that carbon dioxide is more soluble to water compared to oxygen. Therefore, the gas membrane layer coated on the top side of the gas permeable transparent sheet may selectively uptake carbon dioxide from air into the pond water and the gas membrane layer coated on the bottom side of the gas permeable transparent sheet may selectively expel oxygen from pond water into air. To increase photosynthesis and growth of algae, gas permeable membrane layers that are highly selective to carbon dioxide and oxygen are required.

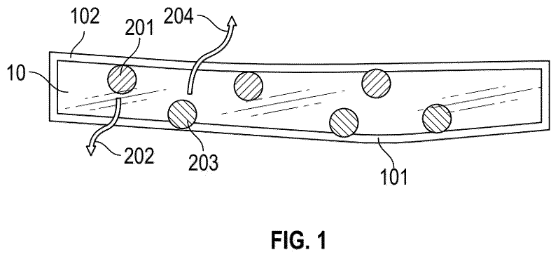

FIG. 1 is a cross-sectional view of the gas permeable transparent sheet 10 coated with gas permeable membrane layers 101 and 102 in accordance with one or more embodiments of the present disclosure. Specifically, the bottom side of gas permeable transparent sheet 10 is coated with the gas permeable membrane layer 101 and the top side of gas permeable transparent sheet 10 is coated with the gas permeable membrane layer 102. The gas permeable transparent sheet 10 coated with the gas permeable membrane layers 101 and 102 may exchange carbon dioxide 201 and oxygen 203 selectively. For example, the gas permeable membrane layer 102 coated on the top side of the gas permeable transparent sheet 10 allows uptake 202 of carbon dioxide and the gas permeable membrane layer 101 coated on the bottom side of the gas permeable transparent sheet 10 allows expulsion 204 of oxygen 203.

In one or more embodiments, the gas permeable membrane layer may have a thickness of from 2.0 μm to 5.0 μm. For example, the gas permeable membrane layer of one or more embodiments may have a thickness in a range having a lower limit of any one of 2.0, 2.1, 2.2, 2.3, 2.4, 2.5, 2.6, 2.7, 2.8, 2.9, 3.0, 3.5, and 4.0 μm, and having an upper limit of any one of 3.0, 3.1, 3.2, 3.3, 3.4, 3.5, 3.6, 3.7, 3.8, 3.9, 4.0, 4.5, and 5.0 μm where any lower limit may be paired with any mathematically compatible upper limit.

As discussed above, suitable gas permeability is required for carbon dioxide uptake and oxygen expulsion such that it may be advantageous to increase photosynthesis and growth of algae in algal ponds.

In one or more embodiments, the composition of the gas permeable membrane layer disclosed herein may include a polymer. Examples of such polymers include, but are not limited to, polyhydroxyalkanoates (PHA), polylactic acid (PLA), chitosan, cellulose, poly[bis(2-2 (methoxyethoxy)ethoxy)]phosphazene, and polytrimethylsilylpropyne.

As noted above, embodiments disclosed herein may contain light-shifting materials to enhance photosynthesis by converting light being transmitted through the shade to more favorable frequencies for photosynthesis. Therefore, in one or more embodiments, the composition of the gas permeable membrane layer may further include a light frequency-shifting material. Examples of light frequency-shifting material disclosed herein may include, but are not limited to, a quantum dot, a fluorescent protein, and combinations thereof.

In one or more embodiments, a quantum dot may be a core/shell nanoparticle including an inner core. Examples of the material that makes up the inner core may include, but are not limited to, CuInS₂, Zn₃P₂, GaP, GaAs, GaSb, ZnS, ZnSe, ZnTe, CdSe, CdS, CdTe, PbS, PbSe, PbTe, and combinations thereof.

In one or more embodiments, a nanocrystal quantum dot may be a core/shell nanoparticle including an outer shell. Examples of the material that makes up the outer shell may include, but are not limited to, ZnS, ZnSe, ZnTe, CdS, CdSe, CuGaS, GaP, and combinations thereof.

In one or more embodiments, examples of fluorescent protein as light frequency-shifting materials may be a green fluorescent protein (GFP) and a red fluorescent protein (RFP). Indeed, any fluorescent protein that absorbs light containing blue light and emits light containing red light is suitable.

### Shade System

As discussed above, the shade system includes a smart algae shade containing a gas permeable transparent sheet coated with a gas permeable membrane layer. The “shade” of the present disclosure refers to a gas permeable transparent sheet coated with a gas permeable membrane layer on each side of the gas permeable transparent sheet as a whole.

In one or more embodiments, the shade system may have an overall gas permeability of 1 Barrer to 100 Barrers. For example, the shade system of one or more embodiments may have an overall gas permeability in a range having a lower limit of any one of 1, 2, 3, 4, 5, 6, 7, 8, 9, 10, 15, 20, 25, 30, 35, 40, 45, 50, 60, 70, and 80 Barrers, and having an upper limit of any one of 30, 40, 45, 50, 55, 60, 65, 70, 75, 80, 85, 90, 91, 92, 93, 94, 95, 96, 97, 98, 99, and 100 Barrers, where any lower limit may be paired with any mathematically compatible upper limit.

The gas permeability may be different for different gases of interest. For example, O₂, CO₂, and N₂ are required for algae growth, and the gas permeability of each gas may range from about 10-15 Barrers for O₂, from about 1-10 Barrers for N₂ and from about 15-100 Barrers for CO₂, respectively.

For example, the shade system of one or more embodiments may have an O₂ permeability in a range having a lower limit of any one of 10, 10.2, 10.4, 10.6, 10.8, 11.0, 11.2, 11.4, 11.6, 11.8, 12, 12.2, 12.4, 12.6, 12.8, 13.0, and 14.0 Barrers, and having an upper limit of any one of 11, 12.0, 12.2, 12.4, 12.6, 12.8, 13.0, 13.2, 13.4, 13.6, 13.8, 14, 14.2, 14.4, 14.6, 14.8, and 15 Barrers, where any lower limit may be paired with any mathematically compatible upper limit.

For example, the shade system of one or more embodiments may have an CO₂ permeability in a range having a lower limit of any one of 15, 16, 17, 18, 19, 20, 25, 30, 35, 40, 45, 50, 55, 60, 65, 70, 75 and 80 Barrers, and having an upper limit of any one of 35, 40, 45, 50, 55, 60, 65, 70, 75, 80, 85, 90, 95, 96, 97, 98, 99, and 100 Barrers, where any lower limit may be paired with any mathematically compatible upper limit.

For example, the shade system of one or more embodiments may have an N₂ permeability in a range having a lower limit of any one of 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, and 7.0 Barrers, and having an upper limit of any one of 4.0, 4.5, 5.0, 5.5, 6.0, 6.5, 7.0, 7.5, 8.0, 8.5, 9.0, 9.1, 9.2, 9.3, 9.4, 9.5, 9.6, 9.7, 9.8, 9.9 and 10 Barrers, where any lower limit may be paired with any mathematically compatible upper limit.

As discussed above, the shade has a gas permeable transparent sheet coated with a gas permeable membrane layer on each side (top side and bottom side) of the gas permeable transparent sheet. In one or more embodiments, the shade may have a total light transmittance of 26 μmol photons·m⁻²·s⁻¹ to 400 μmol photons·m⁻²·s⁻¹, which allows visible light to pass through the shade. Shades having a total light transmittance in such range allows transmission of red light and blue light used for algae growth.

The shade systems herein float atop the pond, thereby limiting an exposed surface area of the pond. The shade systems thus have a buoyancy such that a totality or a portion of the shade system is above the water level in the pond. In other words, partial submersion of the shade system is acceptable in one or more embodiments herein. In one or more embodiments, the shade may have a buoyant force that is equal to or larger than the gravitational force acting on the shade such that the shade float in the pond water.

To promote the floating of the shade system on the surface of the pond, the shade system according to embodiments herein has an overall density less than that of the water in the pond. In one or more embodiments the shade may have a density at or less than about 1.0 g/cm³, such as less than about 0.95 g/cm³, or less than about 0.90 g/cm³, or less than about 0.85 g/cm³, or less than about 0.80 g/cm³, or less than about 0.75 g/cm³, or less than about 0.70 g/cm³, or less than about 0.65 g/cm³, or less than about 0.60 g/cm³, or less than about 0.55 g/cm³, or less than about 0.50 g/cm³, or less than about 0.45 g/cm³, or less than about 0.40 g/cm³, or less than about 0.35 g/cm³, or less than about 0.30 g/cm³, or less than about 0.25 g/cm³, or less than about 0.20 g/cm³, or less than about 0.15 g/cm³, or less than about 0.10 g/cm³.

Algal shades according to embodiments herein may have any shape or size suitable for use on a particular pond. For example, algal shades can be in the shape of a lily pad, oval, circular, rectangular, square, or other various shapes. Embodiments herein contemplate the shade being in the form of an elongated rectangle, as may be used to cover a majority of a straight portion of a “racetrack” algal pond. Other embodiments herein contemplate use of multiple lily pad shaped algal shades for covering a substantial portion of a surface of an algal pond such as tanks or circular ponds.

In one or more embodiments, elongated rectangle shaped shades that may be used in “racetrack” algal pond may have any reasonable size with respect to each application. Examples of the size of the continuous sheet may be 6 feet×12 feet to 6 feet×60 feet. In one or more embodiments, the elongated rectangle shaped shades may have an aspect ratio (length to width) in a range from 1:2 to 1:10.

In one or more embodiments, lily pad shaped algal shades may have any reasonable diameter with respect to each application. Examples of the diameter of the lily pad shaped algal shades may be 8 cm to 30 cm. For example, the lily pad shaped algal shades of one or more embodiments may have a diameter in a range having a lower limit of any one of 8.0, 8.5, 9.0, 9.5, 10.0, 10.5, 11.0, 11.5, 12.0, 14.0, 16.0, 18.0, 20.0, 22.0, and 24.0 and having an upper limit of any one of 14.0, 16.0, 18.0, 20.0, 22.0, 24.0, 26.0, 26.5, 27.0, 27.5, 28.0, 28.5, 29.0, 29.5, and 30 where any lower limit may be paired with any mathematically compatible upper limit.

Furthermore, in one or more embodiments, algal shades may have at least one rod attached. The rod may be a housing, a floating rod, or an anchor such that the movement of the modular sheet may be limited in the algal pond. A rod such as a housing or an anchor may be physically connected to the bottom of the algal pond.

In one or more particular embodiments, the floating rod may have a weight that does not impact the buoyancy of the gas permeable modular sheet. Furthermore, a floating rod may have, for example, a cylindrical shape that may be attached to the center of the algal shades. The cylindrical-shaped floating rod may have a diameter in a range of 1.0 cm to 10.0 cm. For example, the cylindrical-shaped floating rod of one or more embodiments may have a diameter in a range having a lower limit of any one of 1.0, 1.2, 1.4, 1.6, 1.8, 2.0, 2.2, 2.4, 2.6, 2.8, 3.0, 3.2, 3.4, 3.6, 3.8, 4.0, 5.0, 6.0, and 7.0, and having an upper limit of any one of 4.0, 5.0, 6.0, 7.0, 7.2, 7.4, 7.6, 7.8, 8.0, 8.2, 8.4, 8.6, 8.8, 9.0, 9.2, 9.4, 9.6, 9.8, and 10.0 where any lower limit may be paired with any mathematically compatible upper limit. Furthermore, in one or more embodiments, the length of the cylindrical-shaped floating rod may be in a range of 2.0 cm to 40.0 cm. For example, the cylindrical-shaped floating rod of one or more embodiments may have a length in a range having a lower limit of any one of 2.0, 3.0, 4.0, 5.0, 6.0, 7.0, 8.0, 9.0, 10.0, 15.0, 20.0, 25.0, and 30.0 and having an upper limit of any one of 10.0, 15.0, 20.0, 25.0, 30.0, 31.0, 32.0, 33.0, 34.0, 35.0, 36.0, 37.0, 38.0, 39.0, and 40.0 where any lower limit may be paired with any mathematically compatible upper limit.

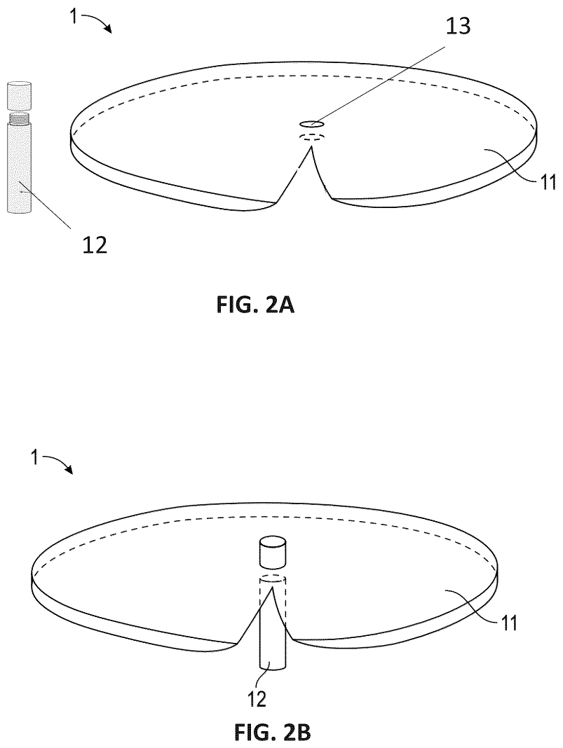

FIGS. 2A and 2B illustrate a shade system 1 of algae cultivation in accordance with one or more embodiments of the present disclosure. As shown in FIGS. 2A and 2B, a shade system 1 may include a lily pad shaped algal shade 11 and at least one rod 12. FIG. 2A shows that the shade may have at least one hole 13 designed, for example, at the center of the lily pad shaped algal shade 11 such that at least one rod 12 can be inserted through the hole 13 of the shade 11. Furthermore, for example, the rod 12 may have a thread part such that the rod 12 can be fixed at the hole 13 of lily pad shaped algal shade 11. As shown in FIG. 2B, at least one rod 12 may be attached to the shade to limit the movement of the lily pad shaped algal shade 11 so that the lily pad shaped algal shade 11 stays in a mostly fixed position on the pond.

Furthermore, the rod 12 may have an adjustable height such that the algal shade is in contact with the surface of pond water. For example, the height of the rod can be adjusted to have the lower portion of the rod (which refers to the portion of the rod that is below the algal shade) to have a longer length than the upper portion of the rod (which refers to the portion of the rod that is above the algal shade).

In one or more embodiments, algae open pond may include one or more shade systems such that the system covers the surface of the algal pond thereby reducing the water evaporation from the algal pond. In one or more particular embodiments, the coverage of pond surface area relative to the total surface area of the pond may be 10% to 80%. For example, the coverage of pond surface area relative to the total surface area of the pond may be in a range having a lower limit of any one of 10, 15, 20, 25, 30, 35, 40, 50, and 60% and having an upper limit of any one of 30, 40, 50, 55, 60, 65, 70, 75, and 80% where any lower limit may be paired with any mathematically compatible upper limit.

In one or more embodiments, one or more sensors may be used to monitor at least one of salinity, pH, water level, and dissolved gas concentration of water. One or more sensors may be embedded entirely or partially in the system, in algal ponds, and/or in control structures.

Specifically, one or more sensors may be attached to the inside or outside of the gas preamble transparent sheet such that the sensor is in contact with the algal pond water.

Alternatively, in one or more embodiments, one or more sensors may be attached to the inside or outside of the lower part of the rod of the system such that the sensor is in contact with the algal pond water. For example, in one or more embodiments, the sensors may be distributed in some of or all of the algal shades of the system for real time monitoring of salinity, pH, and temperature of pond water.

In one or more embodiments, data for salinity, pH, and temperature of pond water may be collected from the sensors and sent to a database in a server that can be accessed over the Internet, which is connected to a media router. The collected data can be displayed on a website dashboard in graphs, illustrations, or numerical data to monitor the ponds in real time.

In one or more embodiments, the system may further include a data transmitter to transmit data collected by one or more sensors to a receiver. A transmitter may be any kind of transmitter that may transmit data to the receiver. In one or more embodiments, the sensors and transmitters may use a power source, such as batteries. Additionally, the batteries may be powered by renewable energy sources, such as solar or wind power.

In one or more embodiments, the transmitted data of salinity, pH, water level, and dissolved gas concentration of water in a pond, that are monitored by the sensor, may be used to adjust at least one of salinity, pH, water level, and dissolved gas concentration of water for effective algae cultivation.

In one or more particular embodiments, the system may further monitor the temperature of the gas permeable membrane layer and transmit data to the receiver such that the temperature of the membrane network may be monitored. In one or more embodiments, the system may further include a microfluidic cooling network for thermal management in the membrane network. The microfluidic cooling network may be installed around or inside the system. When the membrane layer is overheated, the temperature of the membrane layer may be adjusted with the microfluidic cooling network to cool the membrane layer. As used herein, “overheat” refers to a gas permeable membrane layer that exceeds the temperature of 50° C. to 70° C.

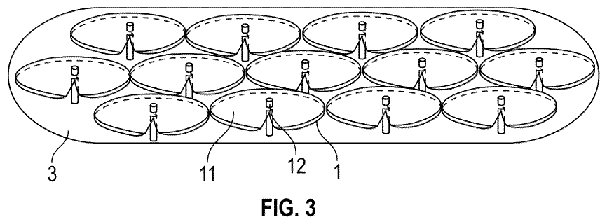

FIG. 3 is a schematic drawing of the algal pond water 3 covered with one or more shade systems 1 of one or more embodiments. The shade system 1 includes a lily pad shaped algal shade 11 and at least one rod 12.

### A Method for Algae Cultivation

In one or more embodiments, the method of algae cultivation includes introducing the system into an algal pond, and cultivating algae in the algal pond.

In one or more embodiments, the method of algae cultivation includes introducing, into an algal pond, a system that contains one or more sensors, monitoring at least one selected from salinity, pH, water level, and dissolved gas concentration of water, adjusting the at least one selected from salinity, pH, water level, and dissolved gas concentration of water based on the results of the monitoring; and cultivating algae in the algal pond.

Embodiments of the present disclosure may provide at least one of the following advantages. The system including a gas permeable transparent sheet may shade the surface of open pond to reduce water evaporation without negatively affecting photosynthesis of algae. The gas permeable transparent sheet allows visible light to pass through and reach the algae in the algal pond such that the visible light can be used for photosynthesis. Furthermore, the gas permeable transparent sheet may be coated with a gas permeable membrane layer. The gas permeable membrane layer allows uptake of carbon and expulsion of oxygen such that it increases photosynthesis of the algae.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A system for algae cultivation, comprising:
an algae shade comprising a gas permeable transparent sheet coated with a gas permeable membrane layer,
wherein the gas permeable membrane layer is coated on a top side of the gas permeable transparent sheet and a bottom side of the gas permeable transparent sheet respectively, and
wherein the gas permeable membrane layer has a thickness of from 1.0 nm to 10,000 nm.

2. The system of claim 1, further comprising at least one rod attached to the gas permeable transparent sheet.

3. The system of claim 1, wherein the system further comprises one or more sensors to monitor at least one parameter selected from salinity, pH, water level, and dissolved gas concentration of water.

4. The system of claim 3, wherein the system further comprises a transmitter that transmits data from the one or more sensors to a receiver.

5. The system of claim 1, wherein the system further comprises a microfluidic cooling network.

6. The system of claim 1, wherein the algae shade has a thickness of 1 μm to 10 μm.

7. The system of claim 1, wherein the algae shade has an aspect ratio ranging of from 1:2 to 1:10.

8. The system of claim 1, wherein the algae shade has a density of 1.0 g/cm³ or less.

9. The system of claim 1, wherein the gas permeable transparent sheet comprises a transparent biocompatible polymer.

10. The system of claim 9, wherein the transparent biocompatible polymer comprises a photo-crosslinkable resin.

11. The system of claim 9, wherein the transparent biocompatible polymer is selected from the group consisting of an acrylamide, a polyurethane, a gelatin, an agar, a collagen, an alginate, a carrageenan, a polysiloxane, and combinations thereof.

12. The system of claim 1, wherein the system has a gas permeability of 1 Barrer to 100 Barrer.

13. The system of claim 1, wherein the gas permeable membrane layer is selected from the group consisting of polyhydroxyalkanoates, polylactic acid, chitosan, cellulose, poly[bis(2-2(methoxyethoxy)ethoxy)]phosphazene, polytrimethylsilylpropyne, low density polyethylene, high density polyethylene, polypropylene, poly(imino-1-oxohexamethylene), and nylon 6.

14. The system of claim 1, wherein the gas permeable membrane layer comprises a light frequency-shifting material selected from the group consisting of a nanocrystal quantum dot, a fluorescent protein, and combinations thereof.

15. The system of claim 14, wherein the nanocrystal quantum dot is a core/shell nanoparticle comprising an inner core selected from the group consisting of CuInS₂, Zn₃P₂, GaP, GaAs, GaSb, ZnS, ZnSe, ZnTe, CdSe, CdS, CdTe, PbS, PbSe, PbTe, and combinations thereof.

16. The system of claim 14, wherein the nanocrystal quantum dot is a core/shell nanoparticle comprising an outer shell selected from the group consisting of ZnS, ZnSe, ZnTe, CdS, CdSe, CuGaS, GaP, and combinations thereof.

17. A method for algae cultivation, comprising:
introducing the system of claim 1 into an algal pond; and
cultivating algae in the algal pond.

18. A method for algae cultivation, comprising:
introducing the system of claim 3 into an algal pond;
monitoring the at least one parameter selected from salinity, pH, water level, and dissolved gas concentration of water in the algal pond;
adjusting the at least one parameter selected from salinity, pH, water level, and dissolved gas concentration of water in the algal pond based on results of the monitoring; and
cultivating algae in the algal pond.
